(19) United States
(12) Patent Application Publication
(10) Pub. No.: US 2025/0078696 A1
(43) Pub. Date: Mar. 6, 2025

(54) System and Method for Calibration of Head Mounted Digital Displays for Medical Imaging

(71) Applicant: Luxsonic Technologies Inc., Saskatoon (CA)

(72) Inventors: Michal Wesolowski, Saskatoon (CA); John Corydon Huffman, Normandy Park, WA (US)

(21) Appl. No.: 18/819,049

(22) Filed: Aug. 29, 2024

Related U.S. Application Data
(60) Provisional application No. 63/535,367, filed on Aug. 30, 2023.

(51) Int. Cl. G09G 3/00; A61B 6/46

(52) U.S. Cl. CPC G09G 3/001; A61B 6/462; G09G 2320/0666; G09G 2320/0693; G09G 2380/08

(57) ABSTRACT

Systems and methods for self-calibration of head-mounted displays are provided. A system includes a photosensor disposed at a digital display screen of the head-mounted display and a processor. The processor is configured to display a test pattern on the digital display screen, determine a correction factor based on luminance values as detected by the photosensor of at least a portion of the displayed test pattern, and apply the determined correction factor to adjust a display driving level of the digital display screen.

### RELATED APPLICATION

This application claims the benefit of U.S. Provisional Application No. 63/535,367,filed on Aug. 30, 2023. The entire teachings of the above application are incorporated herein by reference.

### BACKGROUND

To enable consistent and optimal contrast perception for the viewing of medical images, professional radiology standards apply to the displays on which such images are presented as well as to the ambient environments in which radiological reads are performed. For example, standards for display grayscale characteristics and standards for room lighting often apply.

With regard to greyscale display characteristics, computer monitors on which medical images are displayed typically undergo a calibration process that modifies an existing characteristic curve of the display. The characteristic curve (e.g., a curve representing a relationship between a Luminance Output (LO) and a Digital Driving Level (DDL) of each pixel) is modified such that each DDL is maximally distinguishable from that of the other pixels of the display throughout a luminance range. Such calibration processes are typically applied across all display equipment to ensure that the appearance of a medical image is consistent across similarly-calibrated displays and to ensure diagnostic quality.

A greyscale display calibration process can be performed manually or automatically. The calibration process typically involves the display of a test pattern on the computer monitor for visual inspection by a user, who is then able to adjust display parameters of the monitor, or for measurement by a photometer facing the monitor, with software providing for parameter adjustments based on photometer measurements.

Currently, there is no mechanism to calibrate digital displays found in extended reality head mounted displays for use in medical imaging. There exists a need for improved systems and methods for calibrating medical imaging displays in these types of devices.

### SUMMARY

Embodiments disclose systems and methods for calibration of head mounted displays, such as virtual reality headsets, augmented reality headsets, and mixed reality headsets. The systems and methods can be used to enable such head mounted displays to conform to medical imaging display standards.

An embodiment is directed toward a self-calibrating display system that includes a photosensor disposed at a digital display screen of a head-mounted display and a processor. The processor is configured to display a test pattern on the digital display screen, determine a correction factor based on luminance values as detected by the photosensor of at least a portion of the displayed test pattern, and apply the determined correction factor to adjust a display driving level of the digital display screen.

In an embodiment, the processor is further configured to compare a generated characteristic curve of the digital display screen with a standard display function to determine the correction factor. The standard display function may be a greyscale standard display function for medical imaging displays. Optionally, a calibration record can be generated. The calibration record may be generated to store information associated with the calibration of the display system and may include, for example, the calibration settings and/or calibration history for the sensor. The calibration record may be stored, for example, on the device. An advantage of generating and/or storing the calibration record is that the device may not need to be calibrated upon each use.

According to an embodiment, the head-mounted display is a virtual reality headset, augmented reality headset, or mixed reality headset. The digital display screen can include a lens-facing surface and a rear-facing surface (opposing surface), where the photosensor can be disposed at either or both surfaces. The photosensor can comprise one, two, or more photosensors, each of which can be associated with a distinct display screen (or distinct portion thereof) of the head-mounted display. The digital display screen can comprise one, two or more digital display screens, and each of the photosensors can be associated with a distinct one of the display screens.

In an embodiment, the processor can be further configured to determine a color correction factor based on color values as detected by the photosensor of at least a portion of the displayed test pattern and apply the determined color correction factor to adjust a color temperature of the digital display screen.

Another embodiment is directed toward a method for self-calibrating a display system that includes displaying a test pattern on a digital display screen of a head-mounted display and determining a correction factor based on luminance values as detected by a photosensor disposed at the digital display screen. The luminance values are detected from at least a portion of the displayed test pattern. The method further includes applying the determined correction factor to adjust a display driving level of the digital display screen. Optionally, the method can include generating a calibration record.

The method can further include comparing a generated characteristic curve of the digital display screen with a standard display function to determine the correction factor. The standard display function may be a greyscale standard display function for medical imaging displays.

In an embodiment, the head-mounted display is a virtual reality headset, augmented reality headset, or mixed reality headset. The digital display screen can include a lens-facing surface and a rear-facing surface, where the photosensor can be disposed at either or both surfaces. The photosensor can comprise one, two, or more photosensors, each of which associated with a distinct display (or a distinct portion of a display screen) of the head-mounted display. The digital display screen can comprise one, two, or more digital display screens, and each of the photosensors can be associated with a distinct one of the display screens.

The method can further include determining a color correction factor based on color values as detected by the photosensor of at least a portion of the displayed test pattern and applying the determined color correction factor to adjust a color temperature of the digital display screen.

### BRIEF DESCRIPTION OF THE DRAWINGS

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIGS. 1A-1F illustrate schematics of auto-calibrating head mounted displays utilizing one photosensor, according to an embodiment.

FIGS. 2A-2D illustrate schematics of auto-calibrating head mounted displays utilizing more than one photosensor, according to an embodiment.

FIG. 3 illustrates a schematic of an auto-calibration system for a head-mounted display, according to an embodiment.

FIG. 4 is an example test pattern display in a head-mounted display calibration process.

FIG. 5 is a flowchart of an example calibration process, according to an embodiment.

### DETAILED DESCRIPTION

A description of example embodiments follows.

Virtual reality (VR) systems, including extended reality (XR) and augmented reality (AR) systems, make use of head mounted displays (HMDs) to present a virtual, three-dimensional (3D) environment to a user. Recently, VR technologies have been adapted and extended to support various clinical applications, including medical imaging review procedures. Examples of systems and methods providing for virtual medical imaging reading room environments are described in International Pub. No. WO2022/047261A1, the entire teachings of which are incorporated herein by reference.

Traditionally, medical imaging review procedures are conducted in radiology read rooms, with images being presented on one or more computer monitors. The implementation of a read room environment in a virtual reality system can provide for several advantages. For example, whereas a read room is a physical location in which a reader must be present and requires a physical space large enough to accommodate not only the reader but also hardware consisting of computer workstations and monitors, a virtual reality system can effectively create a read room environment in any physical location and with a significantly more compact hardware footprint. Virtual reality can also provide for improved visualization and interaction with complex environments.

A challenge of implementing a read room environment in a virtual reality system is providing for virtual imaging displays that meet professional radiology standards, such as greyscale calibration requirements. Standard calibration devices and processes can be unsuitable for use with head-mounted displays. For example, standard hand-held calibration devices are physically adapted to be held up to a screen of a conventional computer monitor and include photosensors adapted to detect light from such larger-format displays. Similarly, photosensor devices configured to be attached to or integrated within a computer monitor are not capable of being attached to or integrated within a head-mounted display given physical format differences. Furthermore, head-mounted displays of virtual reality devices include display screens that are significantly smaller and disposed much closer to a user's eyes than conventional computer monitors. Such display screens are also configured to present a three-dimensional, 360° environment rather than a flat, two-dimensional presentation. As such, display characteristics affecting a user's perception can vary greatly between conventional displays and head-mounted displays.

Embodiments provide systems and methods for enabling automatic calibration of head-mounted displays. These systems and methods can be particularly advantageous for use in medical imaging review contexts.

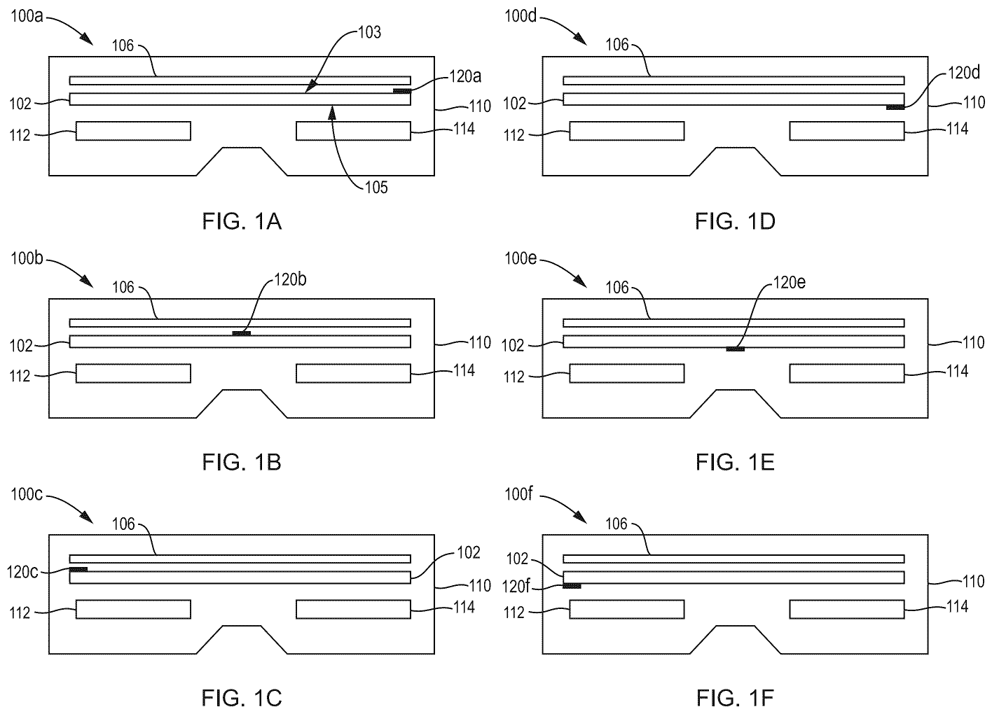

FIG. 1A-1F illustrate examples of self-calibrating head-mounted display systems 100a-100f. Each head-mounted display includes a housing 110, a left lens 112, a right lens 114, a digital display screen 102, and a circuit board 106. Typically, the display screen 102 is disposed between the lenses 112, 114 and the circuit board 106. A photosensor 120a-120f is included within the housing 110. The photosensor 120a-120f is disposed at the digital display screen 102. As used herein, a photosensor disposed “at” a digital display screen means that the photosensor is abutting the display screen, is adjacent to the display screen, or is integrated within the display screen, such that the photosensor can detect light emitted from at least a subset of the pixels of the display screen. For example, the photosensor can be attached to a portion of the display screen or overlaid on the display.

In some embodiments, the head mounted displays 100a-100c illustrate example configurations in which an integrated photosensor 120a-120c is disposed at a rear-facing surface 103 of a display screen 102, relative to lenses 112, 114 of the display. The photosensor 120a-120c is associated with a portion of the display screen 102 presenting to a right lens 114 (see 100a FIG. 1A), associated with a portion of the display screen 102 presenting to both lenses 112 and 114 (see 100b FIG. 1B), and associated with a portion of the display screen 102 presenting to a left lens 112 (see 100c FIG. 1C).

In some embodiments, the head-mounted displays of FIGS. 1D-1F illustrate example configurations in which an integrated photosensor 120d-120f is disposed at a lens- facing surface 105 of a display screen 102, with the photosensor 102d-120f associated with a portion of the display screen 102 presenting to a right lens 114 (see 100d FIG. 1D), associated with a portion of the display screen 102 presenting to both lenses 112 and 114 (see 100e FIG. 1E), and associated with a portion of the display screen 102 presenting to a left lens 112 (see FIG. 1F).

The photosensor can also be disposed at one or more locations relative to lenses 112, 114. For example, as shown in FIG. 1A, a photosensor 120a is disposed at a rear-facing surface 103 (i.e., relative to a wearer) of the display screen 102 and at a location close to, or associated with, a right lens 114 (i.e., as defined with respect to a wearer) of the device. In further examples, a photosensor 120b may be disposed at a location between the left 112 and right 114 lenses of the device 100b, as shown in FIG. 1B. Further, a photosensor 120c may be disposed at a location close to or associated with a left lens 112 of the device, as shown in FIG. 1C. It should be understood that these example photo sensor locations may be used alone or in combination with additional photosensors at different locations.

In yet further examples, a photosensor 120d can be disposed at a lens-facing surface 105 of the display screen 102 and at a location close to or associated with a right lens 114 of the device 100d, as shown in FIG. 1D. A photosensor 120e can be disposed at a lens-facing surface at a location between left 112 and right 114 lenses of the device 100e, as shown in FIG. 1E, and/or a photosensor 120f can be disposed at a location close to or associated with a left lens 112, as shown in FIG. 1F.

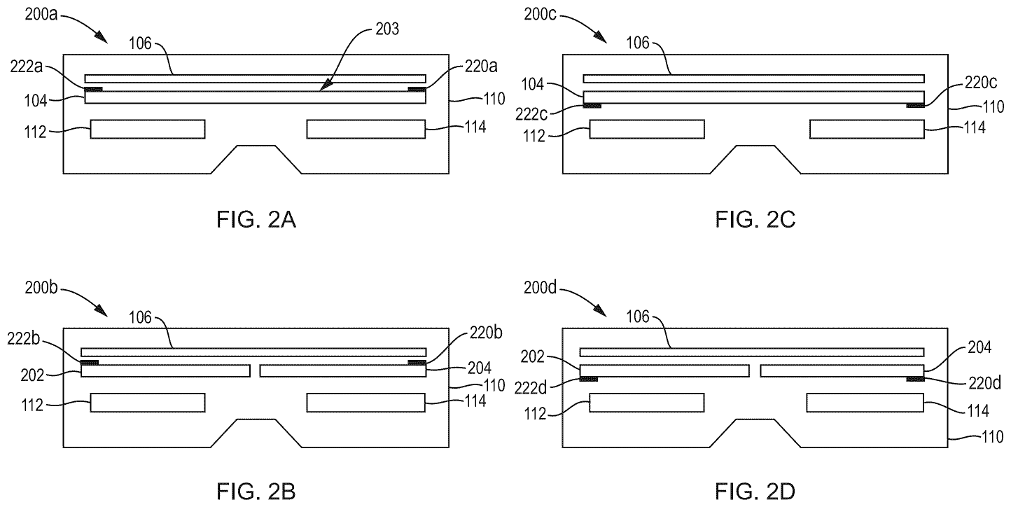

FIGS. 2A-2D illustrate that the head-mounted display may include more than one photosensor, according to some embodiments. For example, as illustrated in 200a of FIG. 2A, photosensors 220a, 222a are disposed at a rear-facing surface 203 of the display screen 104, with a photosensor 220a disposed at a location associated with the right lens 114 and a photosensor 222a at a location associated with the left lens 112 of the device 200a. In another example, as illustrated in 200c of FIG. 2C, photosensors 220c, 222c are disposed at a lens-facing surface 205 of the display screen 104, with a photosensor 220c at a location associated with the right lens 114 and a photosensor 222c at a location associated with the left lens 112 of the device 200c.

FIGS. 2B and 2D illustrate that a head-mounted display may include more than one display screen and can include a photosensor associated with each display screen, according to embodiments. For example, as illustrated in FIG. 2B, the system 200b includes a display screen 202 for display at a left lens 112 and a display screen 114 for display at a right lens 204 of the device 200b. A photosensor 222b is disposed at a rear-facing surface of the display screen 202, and a photosensor 220b is disposed at a rear-facing surface of the display screen 204. In another example, as illustrated in 200d of FIG. 2D, a photosensor 222d is disposed at a lens-facing surface of the display screen 202, and a photosensor 220d is disposed at a lens-facing surface of the display screen 204.

It should be understood that photosensors can be included within a head-mounted display in any combination. For example, the photosensor configurations of any of FIGS. 1A-1F and 2A-2D may be combined such that a head-mounted display includes photosensors at either or both lens-facing and rear-facing surfaces of a display and at any location with respect to lenses of the device.

The photosensors (e.g., photosensors 120a-120f, 220a-220d, 222a-222d) can be configured to detect amounts of light emitted from the display screen (e.g., screen 104) to provide luminance values. A photosensor, as used herein, can comprise a plurality of discrete sensors capable of detecting light. For example, a “photosensor” can comprise an array of photodetection elements. Examples of suitable photodetection elements include photoelectric cells, photovoltaic cells, and photo voltaic transistors. The VR displays used in the HMD typically use a higher pixel density, and may use a smaller pixel pitch, than conventional liquid crystal displays (LCDs). Because the VR display in the HMD is a closed system, there are no ambient light scenarios to consider when calibrating the display. In contrast, for example, a radiologist viewing a diagnostic monitor in a reading room may need to consider the way the ambient light is affecting the display monitor when reading the radiology results. This is not a concern with the VR display in the HMD as it is an inherently isolated system.

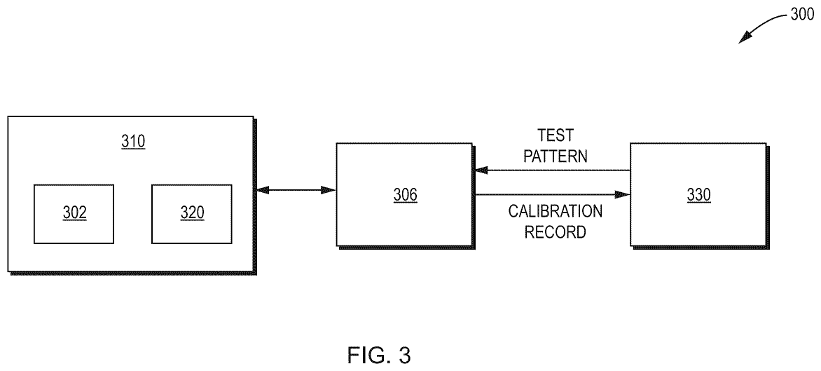

FIG. 3 illustrates a diagram of components of a calibration system for use in embodiments disclosed herein. The system 300 includes a head-mounted display 310, including at least one display screen 302 and at least one photosensor 320. A calibration module 306, executed by a processor, is configured to display a test pattern, retrieved from a data store 330, on the display screen 302. Based on luminance values detected by the photosensor 320, the calibration module 306 determines a correction factor to be applied. For example, the processor can be configured to generate a characteristic curve of the display screen based on measured luminance values and compare the generated curve with a standard display function to determine the correction factor. The correction factor can provide for adjustment of a display driving level (DDL) of the display screen 302. The DDL is a digital value given as an input to a display system to produce a luminance. A processor executing the calibration module 306 can optionally be integrated within a housing of the head-mounted display (e.g., a component of circuit board 106). Optionally, a calibration record can be generated and provided to the data store 330. The calibration record may be generated to store information associated with the calibration of the display system and may include, for example, the calibration settings and/or calibration history for the sensor. The calibration record may be stored, for example, in the data store 330. An advantage of generating and/or storing the calibration record is that the device may not need to be calibrated upon each use.

As used herein, a “standardized display function,” is a function that defines a relationship between input values for pixels of a digital display and a desired output feature (e.g., luminance, color, etc.).

A standardized display function for the display of greyscale images is provided in Part 14 of the Standards for Digital Imaging and Communications in Medicine (DICOM). DICOM Part 14 was developed to provide for a quantitative method of modifying an existing characteristic curve of a display system (e.g., a curve representing a relationship between a Luminance Output (LO) and a Digital Driving Level (DDL) of each pixel) to the Grayscale Standard Display Function (GSDF). The standard provides for maximizing the Just Noticeable Differences (JND) between each DDL and is based on models of human perception. The standard describes a relationship between LO and DDL values such that a slope of a characteristic curve of the digital display (e.g., of contrast) is proportional to that associated with a JND based on the model.

A mechanism for human calibration for the JND for grayscale can be included. The human visual system can “see” a difference of 1 part in 1000; however, it can only discriminate around 7-8 bits of levels. The mechanism can be constructed to allow an individual user to create a value of interest (VOI) look up table (LUT) that can adjust luminance values so that the viewer can “see” all the varying grayscale patterns. A VOI LUT can transform the modality pixel values which are meaningful for the user or the application. For example, as a person ages, their ability to “see” variations in darkness diminishes. This person would then be able to adjust the luminance values to suit their ability.

The standardized display function utilized by a processor of the provided systems and methods can be, for example, the GSDF. Other standard display functions, including display functions for color display, can alternatively be applied. For example, other test patterns can be used, including but not limited to the Commission Internationale de l'Éclairage (CIE) L*a*b Colour Space display function, a log-linear luminance function, and the Color Standard Display Function (CSDF), an extension of the DICOM GSDF.

The test pattern displayed can vary depending upon the standard display function to be applied. Where the standard display function is the GSDF, the test pattern can be the monochrome test pattern published by the Society of Motion Picture and Television Engineers (SMPTE). Other suitable test patterns include the National Equipment Manufacturer Association's (NEMA) test patterns, from the American Association of Physicists in Medicine (AAPM), report “assessment” ASSESSMENT OF DISPLAY PERFORMANCE FOR MEDICAL IMAGING SYSTEMS.[1]

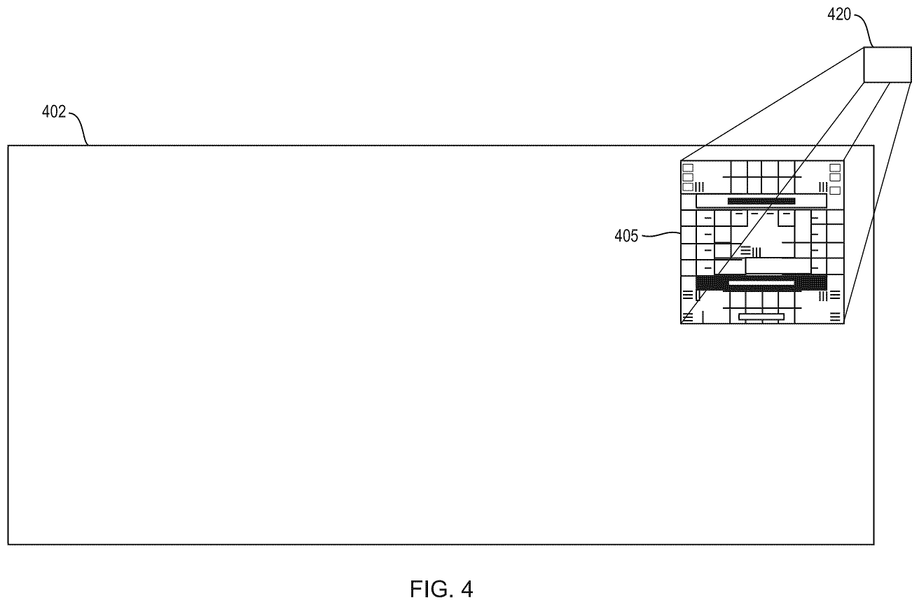

FIG. 4 illustrates that the test pattern can be displayed on a portion of the display screen. As shown in FIG. 4, the test pattern 405 is displayed on a portion of a display screen 402 that correlates to a measurement field of a photosensor 420. For example, for a calibration process using the GSDF, the measurement field can be 10% of a total number of pixels of the display screen 402. While a measurement field of 10% is suitable for the calibration process using the GSDF, a larger or smaller percentage of the total number of pixels on the display screen may be used for suitable applications.

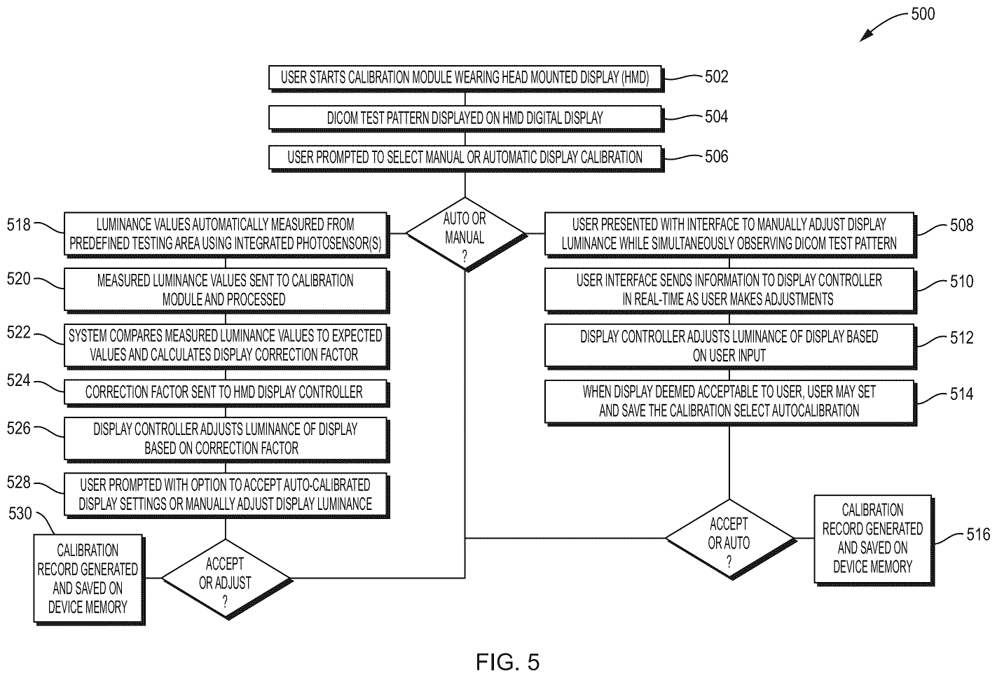

FIG. 5 is a flowchart illustrating a calibration process for embodiments disclosed herein. The process 500 can begin with a user initiating calibration while wearing a head- mounted display (HMD) 502, upon which initiation a test pattern (e.g., a DICOM test pattern) is displayed on a screen of the HMD 504. Optionally, a user can be prompted to select between manual and automatic calibration 506. An option for manual calibration can be included. During manual calibration a user is presented with an interface to manually adjust display parameters while observing the test pattern 508 and such adjustments are provided to a display controller in real-time 510 to adjust a luminance of the display based on user input 512. The user may either accept the manual calibration or move to an autocalibration process 514. If acceptable, a calibration record is stored 516.

During self-calibration, luminance values are automatically measured from the testing area using a photosensor integrated within the HMD 518, and measured luminance values are processed by a calibration module 520. The calibration module compares the measured luminance values to expected values (e.g., based on a standardized displayed function) and calculates a correction factor 522. The correction factor is provided to the display controller 524, and luminance of the display is adjusted accordingly 526. The user may then either accept the autocalibration or move to a manual calibration process 528. If the autocalibration is accepted, a calibration record is stored 530.

The self-calibration process can include causing the display drivers of the digital display to step through a series of DDLs, for example, from minimum to maximum values. At each step, luminance measures can be obtained to generate a characteristic curve of the display. If the luminance measures, or other performance measures, of certain pixels in the display do not fall appropriately along the curve, a correction factor can be applied to those pixels. The correction factor can be calculated based on the difference between the standard display function and the pixels which need correction. These correction factors and the modifications can be determined based on the DICOM standards.[2]

### REFERENCES

[1] Samei E, Badano A, Chakraborty D, Compton K, Cornelius C, Corrigan K, Flynn MJ, Hemminger B, Hangiandreou N, Johnson J, Moxley-Stevens DM, Pavlicek W, Roehrig H, Rutz L, Shepard J, Uzenoff RA, Wang J, Willis CE; AAPM TG18. Assessment of display performance for medical imaging systems: executive summary of AAPM TG18 report. Med Phys. 2005 April;32 (4): 1205-25. doi: 10.1118/1.1861159. PMID: 15895604.
[2] NEMA PS3./ISO 12052, Digital Imaging and Communications in Medicine (DICOM) Standard, National Electrical Manufacturers Association, Rosslyn, VA, USA (available free at http://medical.nema.org/), DICOM PS3.14 2023c-Grayscale Standard Display Function, https://www.dicomstandard.org/standards/view/grayscale-standard-display-function.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

what is claimed is:

1. A self-calibrating display system, comprising:
a photosensor disposed at a digital display screen of a head-mounted display; and
a processor configured to:
display a test pattern on the digital display screen;
determine a correction factor based on luminance values as detected by the photosensor of at least a portion of the displayed test pattern; and
apply the determined correction factor to adjust a display driving level of the digital display screen.

2. The system of claim 1, wherein the processor is further configured to compare a generated characteristic curve of the digital display screen with a standard display function to determine the correction factor.

3. The system of claim 2, wherein the standard display function is a greyscale standard display function for medical imaging displays.

4. The system of claim 1, wherein the head-mounted display is a virtual reality headset, augmented reality headset, or mixed reality headset.

5. The system of claim 1, wherein the digital display screen comprises a lens-facing surface and an opposing surface.

6. The system of claim 5, wherein the photosensor is disposed at the lens-facing surface.

7. The system of claim 5, wherein the photosensor is disposed at the opposing surface.

8. The system of claim 1, wherein the photosensor comprises two or more photosensors, each of the photosensors associated with a distinct display screen of the head-mounted display.

9. The system of claim 8, wherein the digital display screen comprises two or more digital display screens, each of the photosensors associated with a distinct one of the two or more display screens.

10. The system of claim 1, wherein the processor is further configured to:
determine a color correction factor based on color values as detected by the photosensor of at least a portion of the displayed test pattern; and
apply the determined color correction factor to adjust a color temperature of the digital display screen.

11. The system of claim 1, wherein the processor is further configured to generate a calibration record.

12. A method of self-calibrating a display system, comprising:
displaying a test pattern on a digital display screen of a head-mounted display;
determining a correction factor based on luminance values as detected by a photosensor disposed at the digital display screen, the luminance values detected from at least a portion of the displayed test pattern; and
applying the determined correction factor to adjust a display driving level of the digital display screen.

13. The method of claim 12, further comprising comparing a generated characteristic curve of the digital display screen with a standard display function to determine the correction factor.

14. The method of claim 13, wherein the standard display function is a greyscale standard display function for medical imaging displays.

15. The method of claim 12, wherein the head-mounted display is a virtual reality headset, augmented reality headset, or mixed reality headset.

16. The method of claim 12, wherein the digital display screen comprises a lens-facing surface and an opposing surface.

17. The method of claim 16, wherein the photosensor is disposed at the lens-facing surface.

18. The method of claim 16, wherein the photosensor is disposed at the opposing surface.

19. The method of claim 12, wherein the photosensor comprises two or more photosensors, each of the photosensors associated with a distinct display screen of the head-mounted display.

20. The method of claim 19, wherein the digital display screen comprises two or more digital display screens, each of the photosensors associated with a distinct one of the two or more display screens.

21. The method of claim 12, further comprising:
determining a color correction factor based on color values as detected by the photosensor of at least a portion of the displayed test pattern; and
applying the determined color correction factor to adjust a color temperature of the digital display screen.

22. The method of claim 12, further comprising generating a calibration record.
